(19) United States
(12) Patent Application Publication
(10) Pub. No.: US 2010/0197791 A1
(43) Pub. Date: Aug. 5, 2010

(54) PHARMACEUTICALS FOR TRANSDERMAL USE ON ANIMALS

(75) Inventors: Birgitta Pausch, Berlin (DE); Dirk Mertin, Langenfeld (DE); Christel Mueller-Goymann, Braunschweig (DE)

Correspondence Address: BAYER HEALTHCARE LLC, P.O.BOX 390, SHAWNEE MISSION, KS, 66201, US

(73) Assignee: BAYER ANIMAL HEALTH GMBH, LEVERKUSEN (DE)

(21) Appl. No.: 12/670,428

(22) PCT Filed: Jul. 15, 2008
(86) PCT No.: PCT/EP2008/005747

§ 371 Date: Jan. 25, 2010

(30) Foreign Application Priority Data
Jul. 26, 2007 (DE) 10 2007 034 976.0

(51) Int. Cl. A61K 31/196; A61K 31/192; A61P 29/00

(52) U.S. Cl. 514/567; 514/570

(57) ABSTRACT

The invention relates to pharmaceutical preparations for use on animals, which are applied to the fur or the skin of the animal and whose active ingredient subsequently undergoes transdermal absorption.

The invention relates to compositions which increase the transdermal permeability of the skin and thus lead to an increased active ingredient permeability, and to the use of these compositions for the manufacture of medicaments for external use. Transdermal administration of active ingredients is limited greatly by the poor permeability of the skin, especially the stratum corneum. Owing to the very restricted permeability of the skin, only small molecules (molecular mass <500 Da) with lipophilic characteristics are able to overcome this barrier. For all other active ingredients for which transdermal administration is desired it is necessary to improve the transport through the skin. For this reason, active ingredients for transdermal administration are rarely used as pure substance, but are often a constituent of complex formulations consisting of base materials and excipients. Base materials are differentiated into hydrophilic (e.g. water, alcohols) and hydrophobic (e.g. triglycerides, waxes) constituents. Suitable excipients are emulsifiers, gel formers, preservatives and antioxidants.

One possible way of promoting permeation through the skin is to provide preparations for transdermal use with thickeners in order to ensure better adhesion to the skin. This is utilized for pharmaceuticals in the human and in the veterinary sector [WO 04/017998, JP2003095983, U.S. Pat. No. 5,093,133]. Lipophilic components and alcohols can be used to improve the penetration of the stratum corneum. If thickeners are used, evaporation of the alcohol component is desirable in order to achieve better thickening [WO 2005-120473]. In addition, these are usually aqueous systems which may comprise both hydrophilic and hydrophobic base materials [[Brinkmann (2003)], WO 99/022716] and require further additions such as emulsifiers (surfactants) for stabilization [WO 04/017998, WO 01/089469, WO 99/022716, WO 98/051280]. Usually only a small proportion of oil and alcohol component is added [WO 02/096435, EP 428352, EP 91964, WO 92/16237, U.S. Pat. No. 5,093,133]. Other systems in the form of patches operate with coverings which prevent evaporation of volatile constituents of the formulation, and are additionally intended to contribute to improved penetration by bringing about occlusive conditions on the skin [WO 94/09777, EP 1044684].

Active ingredients intended for use in transdermal preparations must have lipophilic properties in order to overcome the lipophilic stratum corneum. For this reason, ionic substances (active ingredient salts) are unsuitable for this purpose and can at most be employed in complex aqueous systems ([Bronaugh, 1984], [Finnin, 1999], [Magnusson, 2004], [Naik, 2000], [Roberts, 2002]). Many active ingredients have only inadequate lipophilic properties or are obtainable only as salts. This makes their use in transdermal formulations difficult and requires a complex composition for the formulation.

The object underlying the invention was therefore to provide formulations for transdermal administrations in which the disadvantages described above for the known formulations are eliminated or diminished. The object was in particular to provide formulations which bring about improved permeation of active ingredient salts and by which the complexity of the formulation can be diminished.

This object is surprisingly achieved by the provision of a pharmaceutical preparation which comprises according to the present invention one or more active ingredient salts and excipients in a mixture consisting of
a. 40%-60% (m/m) of a lipophilic component,
b. 40%-60% (m/m) of an alcohol with the chain length C1-C6 and
c. 0%-10% (m/m) of water.

The invention further relates to the uses defined in the claims.

In a preferred embodiment, the formulation according to the invention comprises at least one active ingredient salt from the group of painkilling substances (analgesics). Analgesics include opioids such as, for example, buprenorphine, codeine, dihydrocodeine, fentanyl, hydromorphone, methadone, morphine, oxycodone, pentazocine, pethidine, piritramide, tilidine, tramadol, and non-opioid analgesics such as, for example, aceclofenac, acemetacin, acetylsalicylic acid, bufexamac, carprofen, celecoxib, deracoxib, diclofenac, etofenamate, etoricoxib, felbinac, flufenamic acid, flunixine, flupirtine, flurbiprofen, ibuprofen, indometacin, ketoprofen, lonazolac, lormnxicam, meclofenamic acid, mefenamic acid, meloxicam, metamizole, mofebutazone, naproxen, nefopam, niflumic acid, oxaprozine, paracetamol, parecoxib, phenazone, phenylbutazone, piroxicam, proglumetacin, propyphenazone, rofecoxib, tepoxalin, tiaprofenic acid, tolfenamic acid, valdecoxib, vedaprofen.

The said active ingredient salts can also be employed in the form of their hydrates, and the invention likewise encompasses enantiomers or racemates. The alkaline earth and alkali metal salts of the said active ingredients are preferred in this connection, especially diclofenac Na, diclofenac K, ketoprofen Na, ketoprofen K, and organic amine salts such as, for example, diclofenac diethylamine.

In one embodiment of the invention, the lipophilic component is selected from the group consisting of neutral oils and fatty acid esters. Neutral oils are for example synthetic triglycerides such as caprylic/capric acid triglycerides, triglyceride mixtures with fatty acids of chain length C8-C12 or other specifically selected natural fatty acids (e.g. Miglyol 810), or, for example, propylene glycol dicaprylate and propylene glycol dicaprate or mixtures thereof (such as, for example, Miglyol 840). Examples of fatty acid esters are isopropyl myristate, isopropyl palmitate, isopropyl stearate, ethyl stearate, hexyl laurate, dipropylene glycol pelargonate, esters of a branched fatty acid of medium chain length with saturated fatty alcohols of chain length C16-C18, caprylic/capric esters of saturated fatty alcohols of chain length C12-C18, oleyl oleate, decyl oleate, ethyl oleate and waxy fatty acid esters such as artificial duck preen gland oil, other esters such as di-n-butyryl adipate, ethyl lactate, dibutyl phthalate, diisopropyl adipate. The group of lipophilic components also includes partial glyceride mixtures of saturated or unsaturated, possibly also hydroxyl group-containing, fatty acids, mono- and diglycerides of C8/C10 fatty acids, liquid paraffins, silicone oils, vegetable oils such as sesame oil, almond oil, castor oil, fatty alcohols such as isotridecyl alcohol, 2-octyldodecanol, cetylstearyl alcohol, oleyl alcohol and fatty acids such as, for example, oleic acid, lauric acid, palmitic acid, stearic acid. The said lipophilic components can be employed alone or as mixture.

In one embodiment of the invention, the alcohol component is an alcohol with a chain length of C1-C6. Alcohols selected from the group consisting of isopropanol, n-propanol, ethanol, methanol, n-butanol, isobutanol, tert-butanol, n-pentanol, n-hexanol, propylene glycol, glycerol and mixtures thereof are preferred.

In a preferred embodiment, the lipophilic component is selected from the group of fatty acid esters or of neutral oils and leads to pharmaceutical preparations which comprise one or more active ingredient salts and excipients in a mixture consisting of
a. 40%-60% (m/m) of a fatty acid ester or of a neutral oil,
b. 40%-60% (m/m) of an alcohol with the chain length C1-C6 and
c. 0%-10% (m/m) of water.

The invention preferably relates to pharmaceutical preparations which comprise one or more active ingredient salts and excipients in a mixture consisting of
a. 40%-60% (m/m) of a fatty acid ester or of a neutral oil and
b. 40%-60% (m/m) of an alcohol with the chain length C1-C6.
In a particularly preferred embodiment of the invention, the alcohol is isopropanol.

The invention particularly preferably relates to pharmaceutical preparations which comprise one or more active ingredient salts and excipients in a mixture consisting of
a. 40%-60% (m/m) of isopropyl myristate and
b. 40%-60% (m/m) of isopropanol.

The invention further particularly preferably relates to pharmaceutical preparations which comprise one or more active ingredient salts and excipients in a mixture consisting of
c. 40%-60% (m/m) of Miglyol 840 and
d. 40%-60% (m/m) of isopropanol.

It has emerged that a formulation which comprises approximately equal parts of alcohol and lipophilic component is preferably of a consistency which spreads well, and brings about an unexpectedly high permeation of the active ingredient, especially when the formulation is anhydrous. Use of the term “anhydrous” does not preclude water being present in the formulation in a proportion of up to 5% by weight. The described preparations are preferred when they comprise less than 2% (w/w) of water, particularly preferably less than 1% (w/w).

A mixture of lipophilic component and alcohol each of 45%-55% (m/m) and 0%-10% (m/m) of water is preferably used. A mixture of lipophilic component and alcohol each of 50% (m/m) is particularly preferably used.

In one embodiment of the invention, further excipients which can be added are antioxidants, substances for UV protection, preservatives and viscosity-increasing substances. Examples of antioxidants are fumaric acid, maleic acid, α-tocopherol, ascorbic acid palmitate, butylated hydroxyanisole, butylated hydroxytoluene, propyl gallate. Examples of preservatives are sorbic acid, benzyl alcohol or phenoxyethanol. Examples of viscosity-increasing substances are colloidal silicon dioxide, bentonite, aluminium stearate, zinc stearate, magnesium aluminium silicate, oleyl oleate, cetyl palmitate, yellow or white wax, ethylene/propylene/styrene and butylene/ethylene/styrene copolymers, Carbopols, cellulose derivatives such as ethylcellulose, hydroxypropylcellulose, methylcellulose, polymeric alcohols such as polyvinyl alcohol. The described preparations preferably make do without surfactants as excipients and thus lead to a reduction in the complexity of the preparation. The described preparations particularly preferably make do without excipients which are required to adjust a pH, i.e. which alter the degree of protonation of the active ingredient salt.

The pharmaceutical preparation is applied to the fur or the skin. This entails topical application of a pharmaceutical preparation comprising the appropriate active ingredient to the animal and subsequent penetration thereof into the fur covering. The active ingredient undergoes transdermal absorption. Topical application preferably takes place for example in the form of spraying, pouring on and rubbing in. The preparations according to the invention furthermore do not require an occlusion-forming covering. The low-viscosity formulation rapidly penetrates, owing to its good spreading properties, into the fur covering. The fur covering over the skin subsequently prevents the volatile components evaporating too quickly from the surface of the skin before they are able to exert their penetration-improving effect on the stratum corneum. Topical application of the preparation according to the invention takes place on the part of the body affected by the disorder, preferably on the joint region, in particular the knee joints. The pharmaceutical preparation is preferably used for animals, particularly preferably for horses, dogs and cats.

The invention further relates to the use of a mixture consisting of a lipophilic component and an alcohol of chain length C1-C6 with in each case a proportion of 40%-60% (m/m) and water in a proportion of 0%-10% (m/m), which comprises one or more active ingredient salts and excipients, for the manufacture of a pharmaceutical preparation. In a preferred form, the lipophilic component is a fatty acid ester or a neutral oil. In a further preferred embodiment, the alcohol is isopropyl alcohol. In a particularly preferred embodiment, the lipophilic component is isopropyl myristate or Miglyol 840 and the alcohol is isopropanol. In a further particularly preferred form, the aforementioned mixtures are anhydrous. In a further particularly preferred form, the aforementioned mixtures are used non-occlusively. A mixture of lipophilic component and alcohol each of 45%-55% (m/m), which may comprise 0%-10% (m/m) of water, is preferably used. A mixture of lipophilic component and alcohol each of 50% (m/m) is particularly preferably used.

The manufacture of the preparation according to the invention can be carried out in a manner known to the skilled person. Solutions or suspensions can be prepared by homogeneously mixing the lipophilic component and alcohol, and dissolving or suspending the active ingredient salt in this mixture. The amount of active ingredient salt varies depending on the purpose of use, substance and size of the area of skin onto which the composition is applied. The skilled person is aware how much active ingredient salt is to be employed for a use. The skilled person is aware of whether further excipients need to be added depending on the use of the preparation.

The invention is explained in more detail by the following examples:

### EXAMPLES

### Example 1

Ketoprofen sodium is dispersed to saturation in a mixture of Miglyol 840, isopropyl alcohol (IPA) and water (45:45:10 m/m/m), a mixture of Miglyol 840, isopropyl alcohol (IPA) (50:50 m/m), a mixture of isopropyl alcohol (IPA) and water (50:50 m/m) and water.

The resulting suspensions have a thermodynamic drug activity of 1.

1000 μl portions are applied to dermatomized (700+/−50 μm) horse skin which is clamped in suitable measuring cells with donor and acceptor compartments.

After 3, 6, 9, 12, 15, 18, 21 and 24 hours, the samples are taken out of the acceptor medium (phosphate buffer), and the active ingredient content is investigated by HPLC.

FIG. 1 shows the permeation from the preparations according to the invention.

The permeation curves make clear that permeation from the anhydrous system is superior to the aqueous formulations.

### Example 2

Ketoprofen acid or ketoprofen sodium are dispersed to saturation in analogy to Example 1 in a mixture of isopropyl myristate (IPM) and isopropyl alcohol (IPA) (50:50 m/m) or a mixture of isopropyl myristate (IPM), isopropyl alcohol (IPA) and water (45:45:10 m/m/m). The resulting suspensions have a thermodynamic drug activity of 1.

FIG. 2 shows the permeation of ketoprofen acid and ketoprofen sodium from the preparations according to the invention.

The permeation curves make it clear that the preparation according to the invention is suitable both for hydrophilic and for lipophilic active ingredients.

The highest values can be attained with the sodium salt of the active ingredient in an anhydrous mixture of isopropyl myristate and isopropyl alcohol.

### Example 3

2.5% (m/m) ketoprofen sodium are dissolved in a mixture of isopropyl myristate (IPM), isopropyl alcohol (IPA) and water (45:45:10 m/m/m). A clear solution results.

1000 μl portions are applied to dermatomized (700+/−50 μm) horse skin which is clamped in suitable measuring cells with donor and acceptor compartments.

The same procedure was applied to the following commercial products:

Phardol® pain gel with 2.5% ketoprofen
Togal® Mobil gel with 2.5% ketoprofen
Effekton® gel with 2.5% ketoprofen

(Since these are acrylate gels, the ketoprofen employed here is also in the form of the sodium salt)

After 3, 6, 9, 12, 15, 18, 21 and 24 hours, samples are taken out of the acceptor medium (phosphate buffer), and the active ingredient content is investigated by HPLC.

TABLE 1
Active ingredient flux after application of 1000
μl of the formulation corresponding to Example
3 to dermatomized horse skin (700 +/− 50 μm), n = 3-4

| Composition of solvent | Flux [μg/cm²/h] |
|---|---|
| IPM - IPA - water (45:45:10% m/m/m) | 653 |
| Phardol ® pain gel | 64 |
| Togal ® Mobil gel | 68 |
| Effekton ® gel | 63 |

The data show that distinctly higher values for the active ingredient flux were achievable with the preparation according to the invention than with commercial products having the same active ingredient content.

### Example 4

Diclofenac sodium or diclofenac acid are dispersed to saturation in analogy to Example 2 in a mixture of isopropyl myristate (IPM) and isopropyl alcohol (IPA) (50:50 m/m). The resulting suspensions have a thermodynamic drug activity of 1.

FIG. 3 shows the permeation from the preparations according to the invention.

The permeation curves make it clear that permeation of the salt is superior to that of the acid.

### Example 5

1% or 4% (m/m) of diclofenac sodium are dissolved in a mixture of isopropyl myristate (IPM), isopropyl alcohol (IPA) (50:50 m/m). Clear solutions result.

1000 μl portions are applied to dermatomized (700+/−50 μm) horse skin which is clamped in suitable measuring cells with donor and acceptor compartments.

The same procedure was applied to the following commercial products:

Voltaren® pain gel with 1% diclofenac diethylamine
Voltaren® pain gel with 1% diclofenac diethylamine+3% diclofenac sodium
Diclac® pain gel with 1% diclofenac sodium
Surpass™ gel with 1% diclofenac sodium
Dolaut® gel with 4% diclofenac sodium

After 3, 6, 9, 12, 15, 18, 21 and 24 hours, samples are taken out of the acceptor medium (phosphate buffer), and the active ingredient content is investigated by HPLC.

FIG. 4 shows the permeation from the preparations according to the invention.

The permeation curves show that distinctly higher values for the active ingredient flux were achievable with the preparation according to the invention than with commercial products with the same active ingredient content.

### Example 6

Ketoprofen is dispersed to saturation in mixtures of isopropropyl myristate (IPM) and isopropyl alcohol (IPA) in various ratios of amounts. The resulting suspensions have a thermodynamic drug activity of 1.

1000 μl portions are applied to dermatomized (700+/−50 μm) horse skin which is clamped in suitable measuring cells with donor and acceptor compartments.

After 3, 6, 9, 12, 15, 18, 21 and 24 hours, samples are taken out of the acceptor medium (phosphate buffer), and the active ingredient content is investigated by HPLC.

TABLE 2
Active ingredient flux after application of 1000
μl of the formulation corresponding to Example
6 on dermatomized horse skin (700 +/− 50 μm), n = 3-4

| Composition of solvent | Flux [μg/cm²/h] |
|---|---|
| 100% IPM | 86.7 |
| 80% IPM + 20% IPA | 167.2 |
| 60% IPM + 40% IPA | 202.2 |
| 40% IPM + 60% IPA | 188.3 |
| 20% IPM + 80% IPA | 149.9 |
| 100% IPA | 43.3 |

### Example 7

Ketoprofen is dispersed to saturation in mixtures of Miglyol 840 and isopropyl alcohol (IPA) in various ratios of amounts. The resulting suspensions have a thermodynamic drug activity of 1.

1000 μl portions are applied to dermatomized (700+/−50 μm) horse skin which is clamped in suitable measuring cells with donor and acceptor compartments.

After 3, 6, 9, 12, 15, 18, 21 and 24 hours, samples are taken out of the acceptor medium (phosphate buffer), and the active ingredient content is investigated by HPLC.

TABLE 3
Active ingredient flux after application of 1000
μl of the formulation corresponding to Example
7 on dermatomized horse skin (700 +/− 50 μm), n = 3-4

| Composition of solvent | Flux [μg/cm²/h] |
|---|---|
| 100% Miglyol 840 | 97.0 |
| 80% Miglyol 840 + 20% IPA | 172.5 |
| 60% Miglyol 840 + 40% IPA | 198.0 |
| 40% Miglyol 840 + 60% IPA | 190.2 |
| 20% Miglyol 840 + 80% IPA | 119.8 |
| 100% IPA | 80.5 |

### Example 8

Ketoprofen is dispersed to saturation in a mixture of Miglyol 840 and isopropyl alcohol (IPA) (50:50 m/m). The resulting suspension has a thermodynamic drug activity of 1.

An analogous procedure is applied to mixtures of:

Miglyol 840 and ethanol (50:50 m/m)
Isopropyl myristate (IPM) and isopropyl alcohol (IPA) (50:50 m/m)
Isopropyl myristate (IPM) and ethanol (50:50 m/m)

1000 μl portions are applied to dermatomized (700+/−50 μm) horse skin which is clamped in suitable measuring cells with donor and acceptor compartments.

After 3, 6, 9, 12, 15, 18, 21 and 24 hours, samples are taken out of the acceptor medium (phosphate buffer), and the active ingredient content is investigated by HPLC.

TABLE 4
Active ingredient flux after application of 1000
μl of the formulations from Example 8 on dermatomized
horse skin (700 +/− 50 μm), n = 3-4

| Composition of solvent | Flux [μg/cm²/h] |
|---|---|
| Miglyol 840 - IPA (50:50% m/m) | 275.4 |
| Miglyol 840 - ethanol (50:50% m/m) | 254.9 |
| IPM - IPA (50:50% m/m) | 317.6 |
| IPM - ethanol (50:50% m/m) | 264.3 |

### Example 9

Ketoprofen sodium is dispersed to saturation in analogy to Example 8 in a mixture of Miglyol 840 and isopropyl myristate (IPM) (50:50 m/m). The resulting suspension has a thermodynamic drug activity of 1.

An analogous procedure is applied to mixtures of:

Miglyol 840 and ethanol (50:50 m/m)
Isopropyl myristate (IPM) and isopropyl alcohol (LPA) (50:50 m/m)
Isopropyl myristate (IPM) and ethanol (50:50 m/m)

### Example 10

Ketoprofen sodium is dispersed to saturation in a mixture of liquid paraffin and isopropyl alcohol (50:50 m/m), leaving an insoluble residue. The resulting suspension has a thermodynamic drug activity of 1.

### Example 11

Ketoprofen sodium is dispersed in analogy to Example 10 in a mixture of sesame oil and isopropyl alcohol (50:50 m/m).

### Example 12

Ketoprofen sodium is dispersed in analogy to Example 10 in a mixture of isopropyl myristate and methanol (50:50 m/m).

### Example 13

Ketoprofen sodium is dispersed in analogy to Example 10 in a mixture of isopropyl myristate and butanol (50:50 m/m).

### Example 14

Diclofenac sodium is subjected to a procedure analogous to Examples 9-13.

### Example 15

Diclofenac acid is subjected to a procedure analogous to Examples 6-8.

### FIGURES

FIG. 1: permeation curves of the average cumulative amount of active ingredient (AI [μg/cm²]) permeated through horse skin (n=3-4) over the time in hours (t [h]) from suspensions of ketoprofen Na in:

Miglyol 840—isopropyl alcohol—water 45:45:10% m/m/m (white square □)

Miglyol 840—isopropyl alcohol 50:50% m/m (black square ▪)

Isopropyl alcohol—water 50:50% m/m (black triangle ▴)

and water (black circle )

FIG. 2: permeation curves of the average cumulative amount of active ingredient (AI [μg/cm²]) permeated through horse skin (n=3-4) over the time in hours (t [h]) from suspensions of:

Ketoprofen acid in isopropyl myristate—isopropyl alcohol—water 45:45:10% m/m/m (full line, black triangle ▴)

Ketoprofen sodium salt in isopropyl myristate—isopropyl alcohol—water 45:45:10% m/m/m (full line, black square ▪)

Ketoprofen acid in isopropyl myristate—isopropyl alcohol 50:50% m/m (broken line, white triangle Δ)

Ketoprofen sodium salt in isopropyl myristate—isopropyl alcohol 50:50% m/m (broken line, white square □)

FIG. 3: permeation curves of the average cumulative amount of active ingredient (AI [μg/cm²]) permeated through horse skin (n=3-4) over the time in hours (t [h]) from suspensions of:

Diclofenac Na in isopropyl myristate—isopropyl alcohol 50:50% m/m (black square ▪) and

Diclofenac acid from isopropyl myristate—isopropyl alcohol 50:50% m/m (white square □)

FIG. 4: permeation curves of the average cumulative amount of active ingredient (AI [μg/cm²]) permeated through horse skin (n=3-4) over the time in hours (t [h]) of:

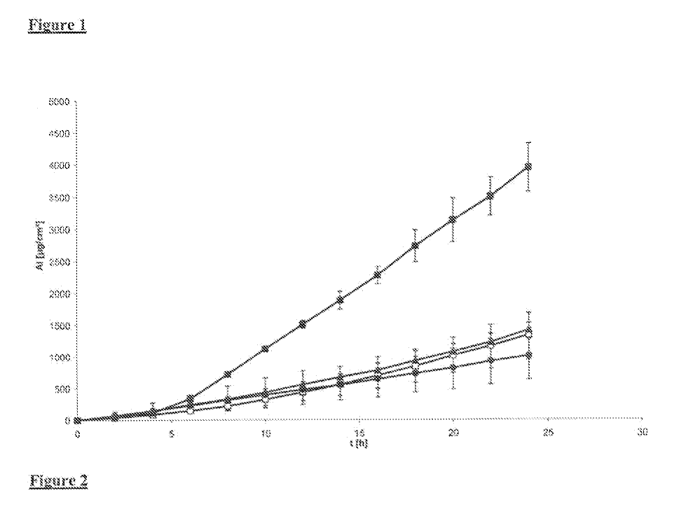

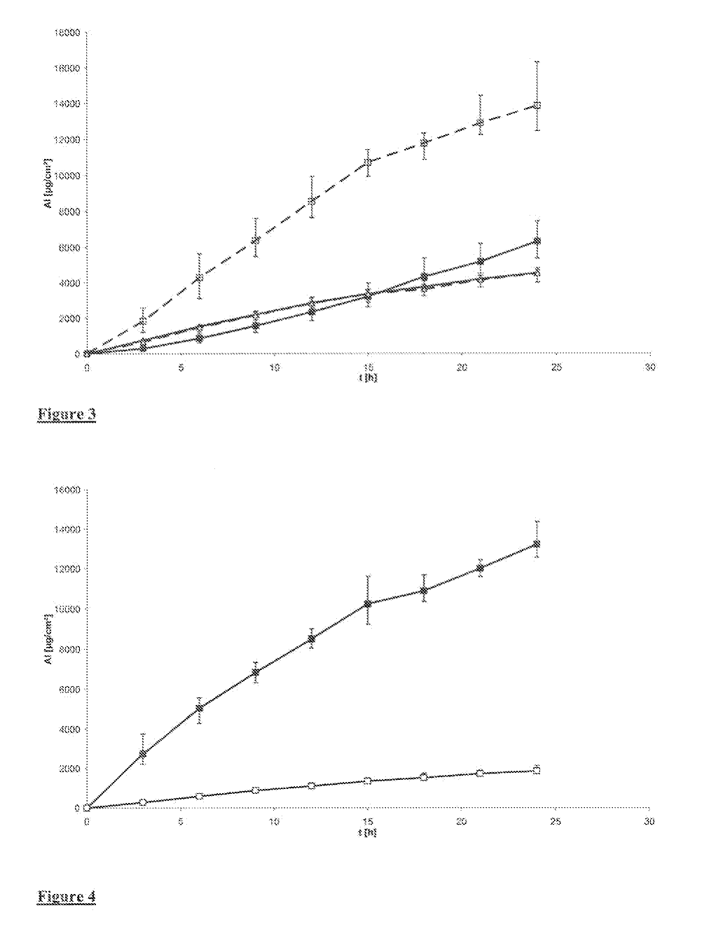

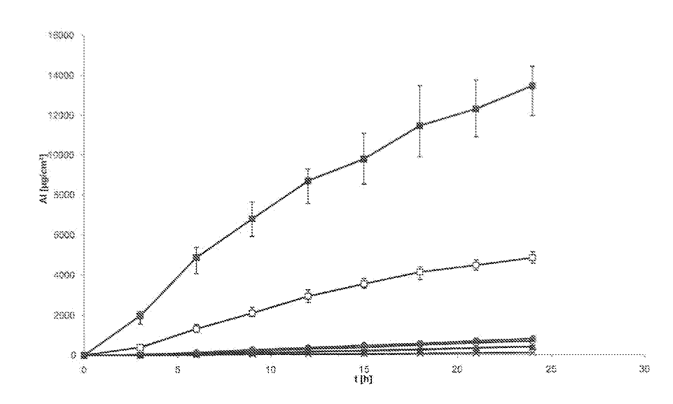

Isopropyl myristate—isopropyl alcohol 50:50% m/m (1% diclofenac Na) (white square □)

Isopropyl myristate—isopropyl alcohol 50:50% m/m (4% diclofenac Na) (black square ▪)

Diclac® pain gel, Hexal (1% diclofenac Na) (white circle ◯)

Surpass™ gel, IDEXX (1% diclofenac Na) (x)

Voltaren® pain gel, Novartis (1% diclofenac diethylamine)(white triangle Δ)

Dolaut®, GiEnne Pharma (4% diclofenac Na) (black circle )

Voltaren® pain gel, Novartis, (1% diclofenac diethylamine, supplemented with 3% diclofenac Na) (black triangle ▴)

### LIST OF REFERENCES

Brinkmann I., Müller-Goymann C. C.: Role of isopropyl myristate, isopropyl alcohol and a combination of both in hydrocortisone permeation across human stratum corneum; Skin Pharmacology and Applied Skin Physiology, 16, 393-404 (2003)
Bronaugh R. L., Congdon E. R.: Percutaneous absorption of hair dyes: Correlation with partition coefficients; The Journal of Investigative Dermatology, 83, 124-127 (1984)
Finnin B. C., Morgan T. M.: Transdermal penetration enhancers: applications, limitations and potential; Journal of Pharmaceutical Sciences, 88, 10, 955-958 (1999)
Magnusson B. M., Pugh W. J., Roberts M. S.: Simple rules defining the potential of compounds for transdermal delivery or toxicity; Pharmaceutical Research 21, 1047-1054 (2004)
Naik A., Kalia Y. N., Guy R. H.: Transdermal drug delivery: overcoming the skin's barrier function; PDTT 3, 9, 318-326 (2000)
Roberts M. S., Cross S. E., Pellett M. A.: Skin transport; Dermatological and Transdermal Formulations, Marcel Dekker, New York; 89-196 (2002), ISBN 0-8247-9889-9

1. A pharmaceutical preparation comprising an analgesic salt and an excipient in a mixture consisting of
a. 40%-60% (m/m) of a lipophilic component,
b. 40%-60% (m/m) of an alcohol with the chain length C1-C6 and
c. 0%-10% (m/m) of water.

2. The pharmaceutical preparation according to claim 1, in which the lipophilic component is a fatty acid ester or a neutral oil.

3. (canceled)

4. The pharmaceutical according to claim 2, in which the analgesic is derived from the group of non-opioid analgesics.

5. The pharmaceutical preparation according to claim 4, in which the non-opioid analgesic is selected from the group consisting of diclofenac sodium, ketoprofen sodium, and mixtures thereof.

6. The pharmaceutical preparation according to claim 5, in which the alcohol is isopropanol.

7. The pharmaceutical preparation according to claim 6, in which the lipophilic component is a fatty acid and wherein the fatty acid ester is isopropyl myristate.

8. The pharmaceutical preparation according to claim 6, in which the lipophilic component is a neutral oil and wherein the neutral oil is Miglyol 840.

9. The pharmaceutical preparation according to claim 8, which is anhydrous.

10. (canceled)

11. The pharmaceutical preparation according to claim 1, in which the alcohol is isopropanol.

12. The pharmaceutical preparation according to claim 1, in which the lipophilic component is a fatty acid and wherein the fatty acid ester is isopropyl myristate.

13. The pharmaceutical preparation according to claim 1, in which the lipophilic component is a neutral oil and wherein the neutral oil is Miglyol 840.

14. The pharmaceutical preparation according to claim 8, which is anhydrous.
